(19) United States
(12) Patent Application Publication
(10) Pub. No.: US 2023/0088685 A1
(43) Pub. Date: Mar. 23, 2023

(54) TOE STIMULUS IMPARTING SUPPORTER

(71) Applicant: GLOBAL ARC, INC., Fukuoka (JP)

(72) Inventors: Kazuma ISHIBASHI, Fukuoka (JP); Tatsumi INOMATA, Fukuoka (JP)

(21) Appl. No.: 17/479,653

(22) Filed: Sep. 20, 2021

(51) Int. Cl. A61F 5/01

(52) U.S. Cl. CPC A61F 5/019; A61F 5/0127

(57) ABSTRACT

Provided is a toe stimulus imparting supporter configured to impart a stimulus to toes in a state where the toes are inserted into the toe stimulus imparting supporter. The toe stimulus imparting supporter includes: a body portion formed of an elastic stretchable member having a strip shape; toe inserting portions through which the toes are insertable, the toe inserting portions formed in both end portions of the body portion in a longitudinal direction of the body portion; and indirect stimulus imparting protruding portions formed on both end portions of the body portion in a protruding manner outward from the body portion.

### BACKGROUND OF THE INVENTION

### Field of the Invention

The present invention relates to a toe stimulus imparting supporter which promotes a motor function and blood circulation and the like by imparting a stimulus to toes.

### Description of the Related Art

Conventionally, there have been known various kinds of training equipment and health equipment which enable a user to promote blood circulation, to improve brain functions and to enhance muscular strength by imparting various stimuli to toes.

Under such circumstances, as equipment which imparts a stimulus to toes, for example, an inter-toe stimulus imparting supporter is disclosed in Japanese Utility Model Laid-open Application No. 03-101929 (patent literature 1). This technique is directed to an inter-toe supporter having stretching property where a portion of a flexible annular body is branched in four such that branched portions are arranged parallel to each other in an annular direction.

A user wears the supporter in such a manner that sub strip portions are disposed between respective toes such that a converging portion is disposed on a sole, an approximately center of a main strip portion is held, the main strip portion traverses an upper portion of a heel from an arc portion by way of a lateral portion of a foot inner side by stretching the main strip portion in a heel direction while being twisted, and the main strip portion reaches the branched portions on an instep of the foot.

By disposing the sub strip portions between the respective toes, a gap is formed between the respective toes, and due to shrinking elasticity of a stretched material, a wearing state of the supporter is maintained, and a pressure is applied between the respective toes in the heel direction.

### SUMMARY OF THE INVENTION

Surely, the inter-toe supporter disclosed in patent literature 1 is referred to as “auxiliary equipment for improving the movement of toes and strengthening the toes by imparting an appropriate stimulus to the respective toes by applying a pressure between respective toes”. Accordingly, it is expected that the inter-toe supporter acquires a certain degree of effect in the promotion of blood circulation, the improvement of brain functions, the improvement of muscular strength, and the like.

However, with respect to toes, the positions of the roots of the toes change obliquely from a first toe to a fifth toe. That is, using an ankle as an origin, a distance between the root of the first toe and the ankle is longer than the distance between the root of the fifth toe and the ankle.

Accordingly, with the use of a single flexible annular body, a strength that the sub strip portions apply between the respective toes is decreased in order from the first toe to the fifth toe so that the inter-toe supporter has a drawback that the above-mentioned effects is halved.

Further, the sub strip portions are not positioned outside the first toe and outside the fifth toe other than between the respective toes so that the inter-toe supporter has a drawback that pressures applied to these two toes are insufficient.

Still further, the converging portion of the inter-toe supporter is positioned on the sole and hence, the inter-toe supporter has a drawback that a user gets a discomfort feeling when the user walks while wearing the inter-toe supporter.

The present invention has been made in view of the above-mentioned circumstances, and it is an object of the present invention to provide a toe stimulus imparting supporter which can apply a substantially uniform pressure to roots between respective toes ranging from a first toe to a fifth toe of a foot and the respective toes ranging from the first toe to the fifth toe of the foot, and can prevent a user from having a discomfort feeling even when the user walks while wearing the toe stimulus imparting supporter.

To achieve the above-mentioned object, the present invention provides a toe stimulus imparting supporter having the following technical features.

(1) According to an aspect of the present invention, there is provided a toe stimulus imparting supporter configured to impart a stimulus to toes in a state where the toes are inserted into the toe stimulus imparting supporter, the toe stimulus imparting supporter including: a body portion formed of an elastic stretchable member having a strip shape; toe inserting portions through which the toes are insertable, the toe inserting portions being formed in both end portions of the body portion in a longitudinal direction of the body portion; and indirect stimulus imparting protruding portions formed on the both end portions of the body portion in a protruding manner outward from the body portion.

(2) In the toe stimulus imparting supporter having the configuration described in (1), a direct stimulus imparting protruding portion is formed on an opening edge of each of the toe inserting portions in a protruding manner from at least a front surface or a back surface of the body portion.

(3) In the toe stimulus imparting supporter having the configuration described in (1), the toe inserting portions are formed such that one toe inserting portion is formed in one end portion of the body portion in the longitudinal direction of the body portion, and two toe inserting portions are formed in the other end portion of the body portion in the longitudinal direction of the body portion.

(4) In the toe stimulus imparting supporter having the configuration described in (1), an opening which is defined by integrally forming two intermediate portions of the body portion in an intersecting manner is formed as a foot inserting portion.

According to the invention described in (1), the toe stimulus imparting supporter includes: the body portion formed of an elastic stretchable member having a strip shape; and the toe inserting portions through which the toes are insertable, the toe inserting portions formed in both end portions of the body portion in the longitudinal direction of the body portion. With such a configuration, when a user merely wears the toe stimulus imparting supporter by inserting the respective toes into the respective toe inserting portions in a state where portions of the body portion intersect with each other from behind an ankle to an instep side of a foot. Accordingly, it is possible to apply an approximately uniform pressure to the roots between the desired respective toes and the desired respective toes ranging from the first toe to the fifth toe.

Further, the body portion is not positioned on the sole even when a user walks while wearing the toe stimulus imparting supporter and hence, there is no possibility that the user has a discomfort feeling.

Further, the indirect stimulus imparting protruding portions are formed on both end portions of the body portion in a protruding manner outward from the body portion. With such a configuration, it is possible to efficiently apply a pressure between the respective toes and the respective toes ranging from the first toe to the fifth toe of the foot from below.

According to the invention described in (2), the direct stimulus imparting protruding portion is formed on the opening edge of each of the toe inserting portions in a protruding manner from at least the front surface or the back surface of the body portion. With such a configuration, it is possible to more efficiently apply a pressure to the roots between the respective toes ranging from the first toe to the fifth toe of the foot.

Further, a pressure can be efficiently applied between the respective toes disposed adjacently to each other ranging from the first toe to the fifth toe of the foot.

According to the invention described in (3), the toe inserting portions are formed such that one toe inserting portion is formed in one end portion of the body portion in the longitudinal direction of the body portion, and two toe inserting portions are formed in the other end portion of the body portion in the longitudinal direction of the body portion. With such a configuration, when a user inserts the first toe and the second toe into the toe inserting portions at the other end portion of the body portion and inserts another desired toe into the toe inserting portion at one end portion, a pressure can be substantially uniformly applied to the roots between the respective toes ranging from the first toe to the fifth toe of the foot and the respective toes.

Further, the left and right sides of the body portion are symmetrically formed and hence, a user can easily wear the toe stimulus imparting supporter on the foot, and the user can also wear the toe stimulus imparting supporter on either the left foot or the right foot.

According to the invention described in (4), the opening which is defined by substantially integrally forming two intermediate portions of the body portion in an intersecting manner is formed as the foot inserting portion. With such a configuration, a user can easily understand a portion of the foot on which the user wears the toe stimulus imparting supporter and how to wear the toe stimulus imparting supporter and hence, the user can wear the toe stimulus imparting supporter intuitively.

### BRIEF DESCRIPTION OF THE DRAWINGS

FIG. 1 is an explanatory view showing a state where a user wears a toe stimulus imparting supporter according to an embodiment on the foot;

FIG. 2A is a plan view of the toe stimulus imparting supporter according to the embodiment;

FIG. 2B is a partially enlarged view of FIG. 2A;

FIG. 2C is a partially cross-sectional view of FIG. 2B;

FIG. 3A is a partially enlarged plan view of the toe stimulus imparting supporter according to the embodiment;

FIG. 3B is a partially cross-sectional view of FIG. 3A;

FIG. 3C is another partially enlarged cross-sectional view of FIG. 3A;

FIG. 3D is another partially enlarged cross-sectional view of FIG. 3A;

FIG. 3E is another partially enlarged cross-sectional view of FIG. 3A;

FIG. 4A is a partially enlarged plan view of the toe stimulus imparting supporter according to the embodiment;

FIG. 4B is a partially cross-sectional view of FIG. 4A;

FIG. 4C is another partially cross-sectional view of FIG. 4A;

FIG. 5A is a partially enlarged plan view of the toe stimulus imparting supporter according to the embodiment;

FIG. 5B is a partially cross-sectional view of FIG. 5A;

FIG. 5C is another partially cross-sectional view of FIG. 5A;

FIG. 6A is a partially enlarged plan view of the toe stimulus imparting supporter according to the embodiment;

FIG. 6B is a partially cross-sectional view of FIG. 6A;

FIG. 6C is a partially enlarged plan view of another toe stimulus imparting supporter according to the embodiment;

FIG. 6D is a partially cross-sectional view of FIG. 6C;

FIG. 7A is a schematic plan view of another toe stimulus imparting supporter according to the embodiment;

FIG. 7B is a schematic plan view of another toe stimulus imparting supporter according to the embodiment;

FIG. 7C is a schematic plan view of another toe stimulus imparting supporter according to the embodiment;

FIG. 7D is a schematic plan view of another toe stimulus imparting supporter according to the embodiment;

FIG. 7E is a schematic plan view of another toe stimulus imparting supporter according to the embodiment;

FIG. 7F is a schematic plan view of another toe stimulus imparting supporter according to the embodiment;

FIG. 8A is a plan view of a toe stimulus imparting supporter according to the embodiment;

FIG. 8B is a plan view of a toe stimulus imparting supporter according to the embodiment;

FIG. 9A is a plan view of a modification of the toe stimulus imparting supporter according to the embodiment; and

FIG. 9B is a partially enlarged view of FIG. 9A.

### DETAILED DESCRIPTION OF THE PREFERRED EMBODIMENTS

The gist of a toe stimulus imparting supporter according to the present invention lies a toe stimulus imparting supporter configured to impart a stimulus to toes in a state where the toes are inserted into the toe stimulus imparting supporter, wherein the toe stimulus imparting supporter includes: a body portion formed of an elastic stretchable member having a strip shape; and toe inserting portions through which the toes are insertable, the toe inserting portions formed in both end portions of the body portion in a longitudinal direction of the body portion. That is, the present invention intends to provide a toe stimulus imparting supporter which can substantially uniformly apply a pressure to roots between desired respective toes of a foot ranging from a first toe to a fifth toe and the respective toes and, at the same time, to prevent a user from having a discomfort feeling or the sole even when the user walks while wearing the toe stimulus imparting supporter.

Hereinafter, one embodiment of a toe stimulus imparting supporter 1a, 1b according to the present invention is described with reference to drawings. In the description made hereinafter, with respect to the structures or parts which are equal on left and right sides or are in left and right symmetry, the same symbols are given in principle, and the structure and parts only on one of the left and right sides are described, and the description of the structures and parts on the other side is omitted when necessary.

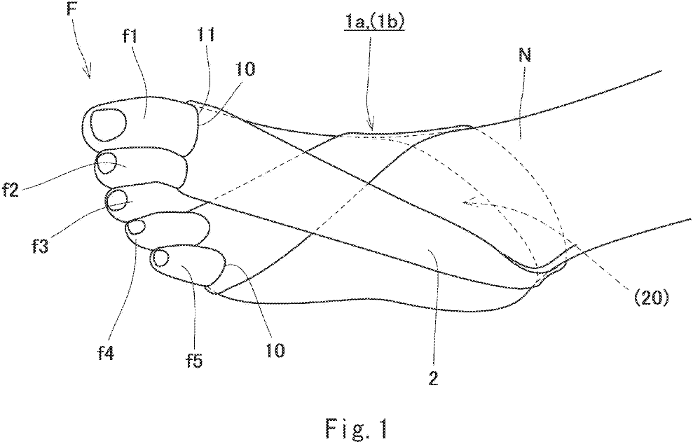

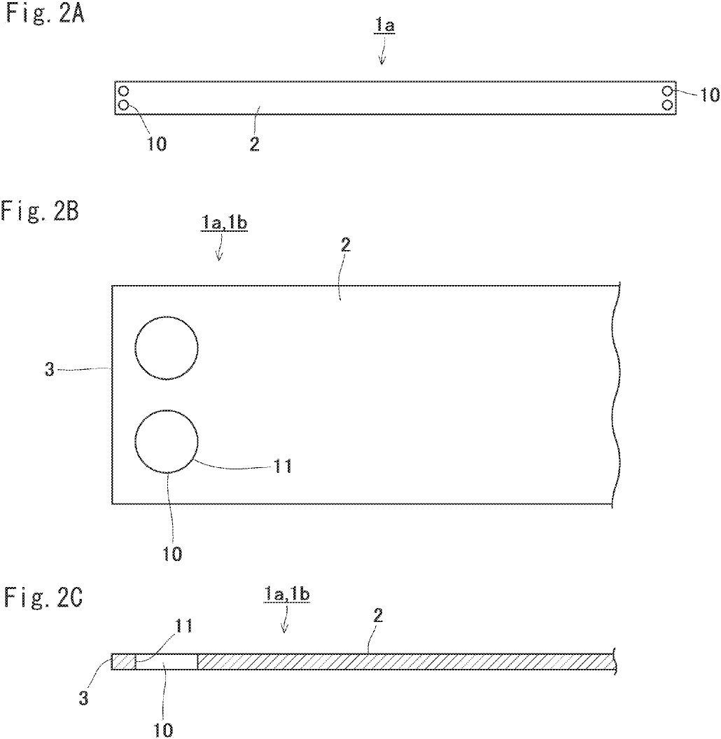

The toe stimulus imparting supporter 1a according to the embodiment of the present invention is described in FIG. 1 and FIG. 2A to FIG. 2C. The toe stimulus imparting supporter 1a imparts a stimulus to toes F in a state where the toes F are inserted into the toe stimulus imparting supporter 1a. The toe stimulus imparting supporter 1a includes: a body portion 2 which is formed of an elastic stretchable member having a strip shape; and one or more toe inserting portions 10 which are formed in both end portions of the body portion 2 (including portions in the vicinity of both end portions) in a longitudinal direction of the body portion 2, extend along an end edge of the body portion 2a (in a short side direction), and through which the toes F are inserted.

In the toe stimulus imparting supporter 1a formed as described above, a first toe f1 and a second toe f2 are inserted into toe inserting portions 10 formed on one end portion of the body portion 2 in the longitudinal direction of the body portion 2 while making the body portion 2 extending from a back side of an ankle N to an instep side and making portions of the body portion 2 intersect with each other, and a fourth toe f4 and a fifth toe f5 are inserted into toe inserting portions 10 formed in the other end portion and hence, a pressure can be substantially uniformly applied to roots between desired respective toes ranging from the first toe f1 to the fifth toe f5 of the foot and the respective toes.

Hereinafter, respective portions of the toe stimulus imparting supporter 1a according to the embodiment are specifically described in detail.

The body portion 2 is formed of an elastic stretchable member having a strip shape and having a predetermined thickness and a predetermined width. Specifically, the body portion 2 is made of a synthetic resin material such as nylon, polyurethan or polyester. However, the body portion 2 may be integrally formed using a material which includes both elasticity and stretchability such as an elastic silicon resin or a rubber material.

With respect to the body portion 2, as shown in FIG. 2A, the toe inserting portions 10 through which the respective toes are inserted are formed in the vicinity of both end portions of the body portion 2 in the longitudinal direction of the body portion 2. The toe inserting portions 10 are formed at two positions in both end portions of the body portion 2 in a state where the toe inserting portions 10 are disposed adjacently to each other along the short side direction of the body portion 2.

As shown in FIG. 2B and FIG. 2C, the toe inserting portion 10 is formed in an approximately circular shape to an extent that the toe can be inserted into the toe inserting portion 10. However, provided that each toe can be inserted, a shape or the like of the toe inserting portion 10 can be flexibly deformed or changed such as being formed into a slit shape by forming a cut in the body portion 2.

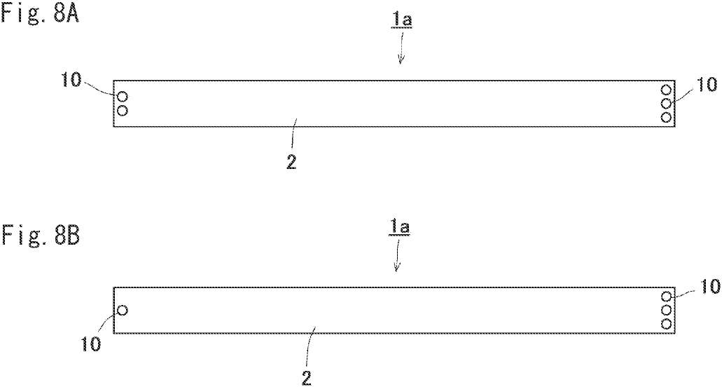

As shown in FIG. 8A, the toe inserting portions 10 are formed at two positions in the vicinity of one end portion of the body portion 2, and the toe inserting portion 10 is formed at three positions in the vicinity of the other end portion of the body portion 2. The number of toe inserting portions 10 is not limited to the present embodiment. As shown in FIG. 8B, the toe inserting portion 10 may be formed at one position in the vicinity of one end portion of the body portion 2, and the toe inserting portion 10 may be formed at three positions in the vicinity of the other end portion of the body portion 2.

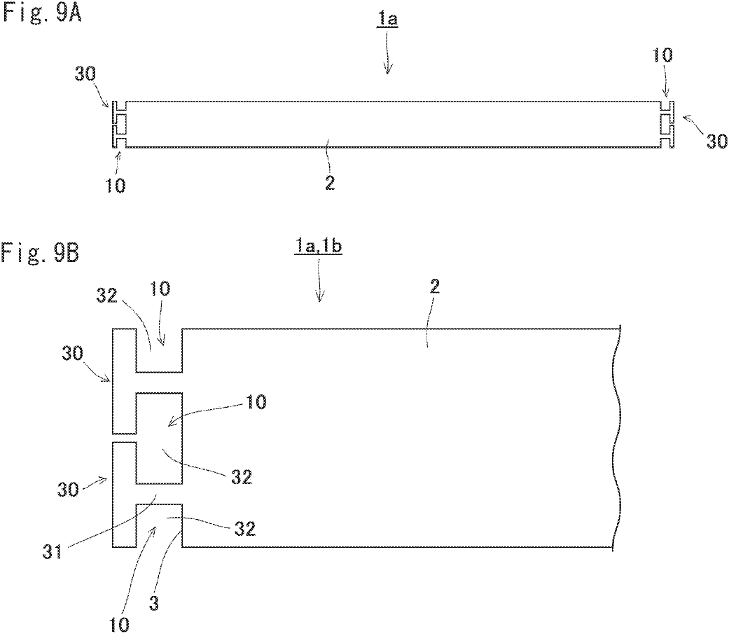

As modifications of the toe stimulus imparting supporter 1a, (1b), as shown in FIG. 9A and FIG. 9B, two toe sandwiching projections 30 having an approximately T shape as viewed in a plan view are formed on an end edge 3 of the body portion 2 in a state where the toe sandwiching projections 30 project toward the outside from the end edge 3, and openings 32 on left and right sides of the toe sandwiching lugs 31 which form the toe sandwiching projections 30 continuously connected to the end edge 3 can also be formed as the toe inserting portions 10.

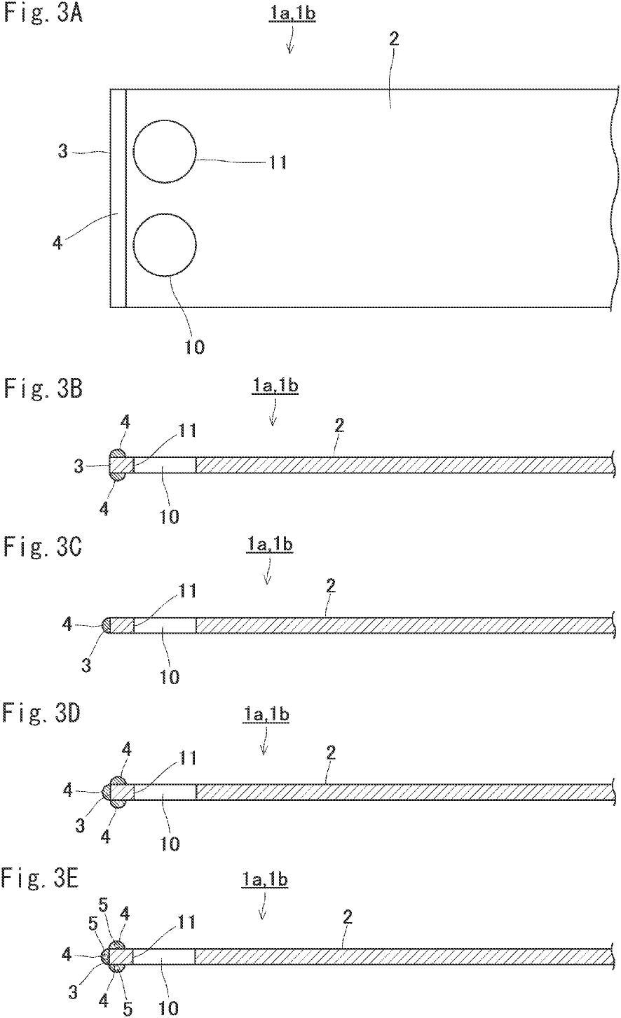

Further, in the embodiment, as shown in FIG. 3A and FIG. 3B, an indirect stimulus imparting protruding portion 4 which protrudes from the body portion 2 and has a predetermined thickness is formed on both end edges 3, 3 which form both end portions of the body portion 2 in the longitudinal direction of the body portion 2.

More specifically, the indirect stimulus imparting protruding portion 4 is positioned at the root of the toe when a user wears the toe stimulus imparting supporter 1a. The indirect stimulus imparting protruding portion 4 is formed in a protruding manner from the body portion 2 by increasing an amount of sewing such that a portion of the body portion 2 where the indirect stimulus imparting protruding portion 4 is formed has a larger thickness than other portions of the body portion 2. The indirect stimulus imparting protruding portion 4 protrudes outward from front and back surfaces of the body portion 2. However, for example, various modifications or changes can be made with respect to the indirect stimulus protruding portion 4 without departing from the gist of the present invention. For example, the indirect stimulus imparting protruding portion 4 are formed by sewing using fine strip-like materials obtained by cutting a material which is substantially the same material as the material for forming the body portion 2.

As shown in FIG. 3C, the indirect stimulus imparting protruding portion 4 may be formed such that the indirect stimulus imparting protruding portion 4 protrudes from an end surface of the body portion 2. As shown in FIG. 3D, the indirect stimulus imparting protruding portion 4 may be formed on the front and back surfaces and the end surface of the body portion 2, that is, from the entire end portion in a protruding manner. The body portion 2 and the indirect stimulus imparting protruding portion 4 may be formed as an integral body using an elastic silicon resin or the like.

Further, as shown in FIG. 3E, a stimulus increasing member 5 made of a rod-shaped material which has both elasticity and stretchability such as an elastic silicon resin or a rubber material may be formed in the indirect stimulus protruding portion 4 along the short side of the body portion 2.

Whether or not the stimulus increasing member 5 is formed in the indirect stimulus imparting protruding portion 4, which part of the indirect stimulus imparting protruding portion 4 the stimulus increasing member 5 is to be formed can be determined as desired.

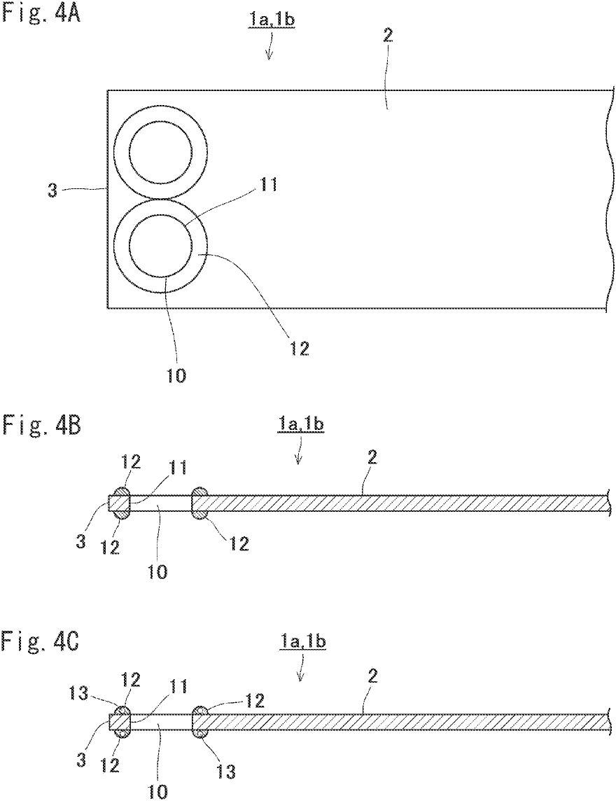

Further, in this embodiment, as shown in FIG. 4A and FIG. 4B, a direct stimulus imparting protruding portion 12 which protrudes with a predetermined thickness larger than a thickness of the body portion 2 is formed on an opening edge 11 of the toe inserting portion 10. That is, the direct stimulus imparting protruding portions 12 are formed such that the direct stimulus imparting protruding portion 12 protrudes from the front and back surfaces of the body portion 2.

Specifically, the direct stimulus imparting protruding portion 12 is a protruding portion which is directly positioned on the root of the toe when a user wears the toe stimulus imparting supporter 1a. The direct stimulus imparting protruding portion 12 is formed in a protruding manner from the body portion 2 by increasing an amount of sewing such that a portion of the body portion 2 where the direct stimulus imparting protruding portion 12 is formed has a larger thickness than other portions of the body portion 2. However, various modifications or changes can be made with respect to the direct stimulus protruding portion 12 without departing from the gist of the present invention. For example, the direct stimulus imparting protruding portion 12 may be, for example, formed by sewing using fine strip-like materials obtained by cutting a material which is substantially the same material as the material for forming the body portion 2.

The direct stimulus imparting protruding portion 12 may be integrally formed with the body portion 2 using an elastic silicon resin or the like.

Further, as shown in FIG. 4C, a direct stimulus increasing member 13 which is made of a circular annular material having both elasticity and stretchability such as an elastic silicon resin or a rubber material is disposed in the direct stimulus imparting protruding portion 12 along an opening edge 11.

Whether or not the direct stimulus increasing member 13 is disposed in the direct stimulus imparting protruding portion 12 and a part in which the direct stimulus imparting protruding portion 12 is disposed are determined as desired.

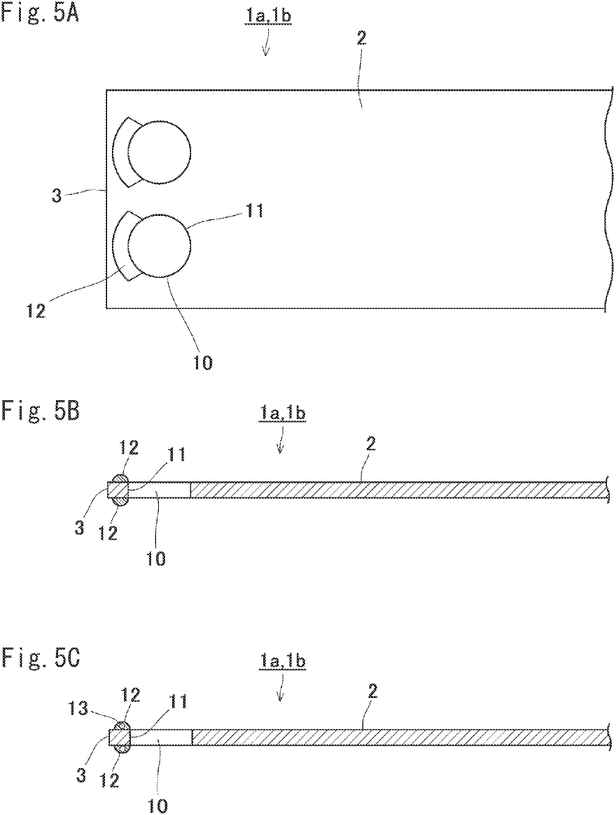

Provided that the direct stimulus imparting protruding portion 12 is directly positioned on the root of the toe when a user wears the toe stimulus imparting supporter 1a, the direct stimulus imparting protruding portion 12 may be formed only on a portion of the opening edge 11 of the toe inserting portion 10 as shown in FIG. 5A and FIG. 5B, for example.

Also in this case, as shown in FIG. 5C, the direct stimulus increasing member 13 can be disposed in the direct stimulus imparting protruding portion 12.

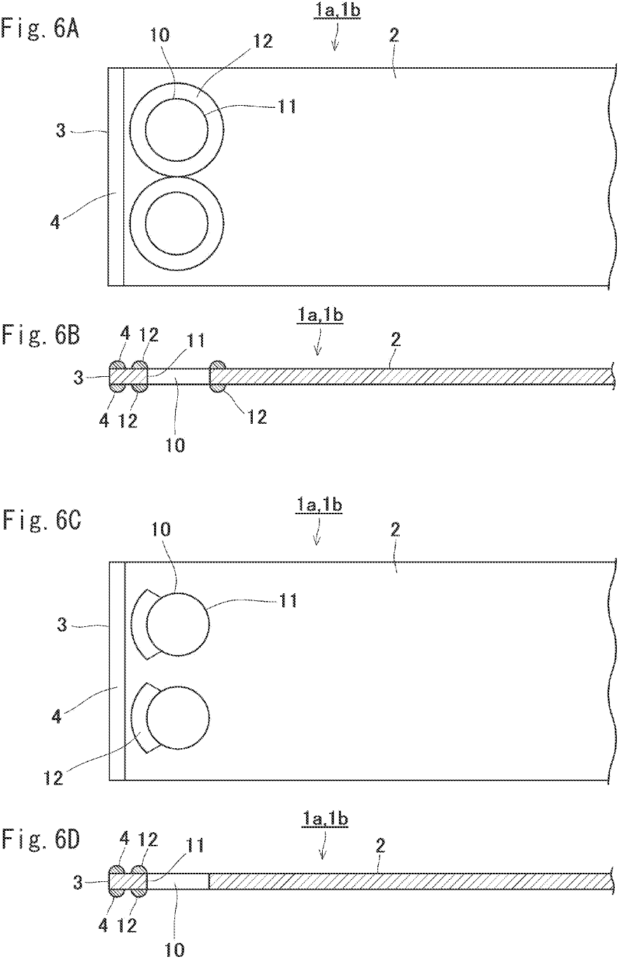

The toe stimulus imparting supporter 1a according to the embodiment may be formed by combining the above-mentioned indirect stimulus imparting protruding portion 4 and the direct stimulus imparting protruding portions 12 as shown in FIG. 6A, FIG. 6B, FIG. 6C, and FIG. 6D.

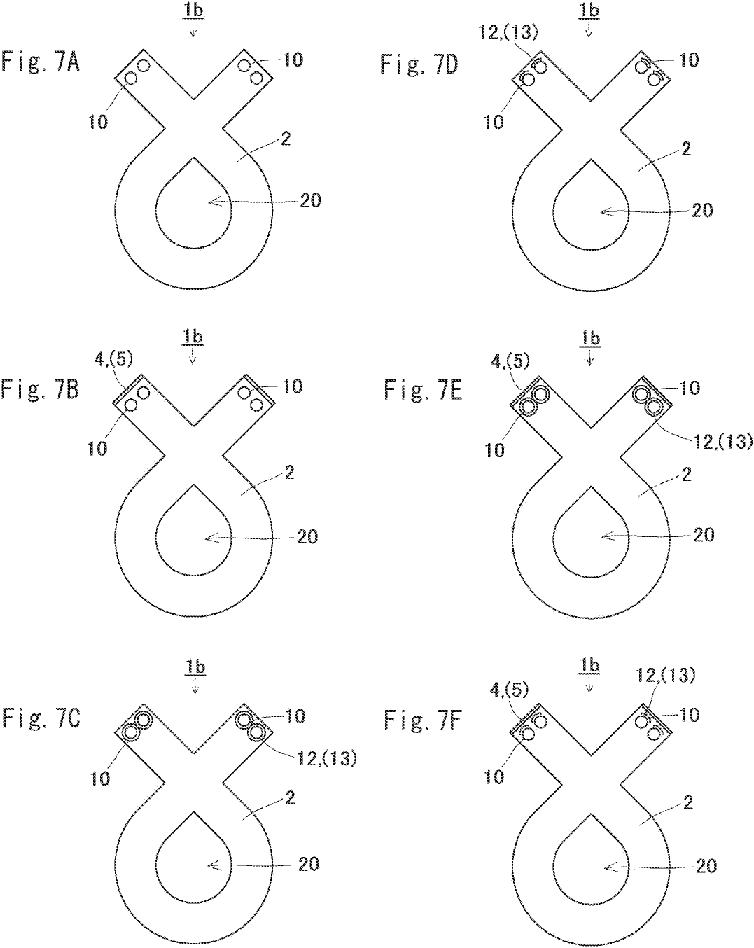

As shown in FIG. 7A, the embodiment also provides the toe stimulus imparting supporter 1b where an opening defined by integrally forming two intermediate portions of the body portion 2 in an intersecting manner is formed as a foot inserting portion 20 through which the ankle N can be inserted.

The portions of the body portion 2 which intersect with each other are integrally connected to each other by sewing or may be formed coplanar with other portions of the body portion 2. Besides such a configuration, the body portion 2 may be formed of one body portion and the other body portion, and the other portion may be insertable into one body portion so that both body portions are slidably movable along the movement of the foot.

Also in this case, as shown in FIG. 7B to FIG. 7F, the above-mentioned indirect stimulus imparting protruding portion 4 and direct stimulus imparting protruding portion 12 may be formed on both end edges 3, 3 which are short sides forming both end portions of the body portion 2 and the opening edge 11 of the toe inserting portion 10, the stimulus increasing member 5 or the direct stimulus increasing member 13 may be disposed in the indirect stimulus imparting protruding portion 4 or the direct stimulus imparting protruding portion 12, or these configurations may be combined with each other.

The toe stimulus imparting supporters 1a, 1b according to the embodiment are formed as described above.

In this manner, the toe stimulus imparting supporter 1a, 1b is the toe stimulus imparting supporter 1a, 1b which imparts a stimulus to the toe F in a state where the toes F are inserted into the toe stimulus imparting supporter 1a, 1b. The toe stimulus imparting supporter 1a, 1b is formed of: the body portion 2 formed of an elastic stretchable member having a strip shape; and one or more toe inserting portions 10 which are formed in the vicinity of both end portions of the body portion 2 in the longitudinal direction of the body portion 2. With such a configuration, when a user merely wears the toe stimulus imparting supporter 1a, 1b by inserting the respective toes into the toe inserting portions 10 in a state where portions of the body portion 2 intersect with each other while directing the body portion 2 from behind the ankle N to the instep side of the foot. Accordingly, it is possible to apply a substantially uniform pressure to the roots between desired respective toes ranging from the first toe f1 to the fifth toe f5 of the foot and the respective toes.

The body portion 2 is not positioned on the sole even when a user walks while wearing the toe stimulus imparting supporter 1a, 1b and hence, the user does not have a discomfort feeling.

By forming the indirect stimulus imparting protruding portion 4 which protrudes with a larger thickness than the body portion 2 on both end edges 3, 3 which are short sides forming both end portions of the body portion 2, it is possible to efficiently apply a pressure to the roots between the respective toes ranging from the first toe f1 to the fifth toe f5 of the foot and the roots of the respective toes from below.

By forming the direct stimulus imparting protruding portions 12 which protrude with a larger thickness than the body portion 2 on the opening edge 11 of the toe inserting portion 10, a pressure can be more effectively applied to the roots of the respective toes ranging from the first toe f1 to the fifth toe f5 of the foot.

A pressure can be efficiently applied between the respective toes disposed adjacently to each other ranging from the first toe f1 to the fifth toe f5 of the foot.

The toe inserting portion 10 is formed at two positions in the vicinity of each of both end portions of the body portion 2. With such a configuration, when a user inserts the first toe f1 and the second toe f2 into the toe inserting portions 10 at one end portion of the body portion 2 and inserts the fourth toe f4 and the fifth toe f5 into the toe inserting portion 10 at the other end portion, a pressure can be substantially uniformly applied to the roots between the respective toes ranging from the first toe f1 to the fifth toe f5 of the foot and the respective toes.

The left and right sides of the body portion 2 are symmetrically formed and hence, a user can easily wear the toe stimulus imparting supporter 1a, 1b on the foot, and the user can also wear the toe stimulus imparting supporter 1a, 1b to either the left foot or the right foot.

By forming the opening which is defined by substantially integrally forming two intermediate portions of the body portion 2 in an intersecting manner as the foot inserting portion 20, a user can easily understand a portion of the foot on which the user wears the toe stimulus imparting supporter 1b and how to wear the toe stimulus imparting supporter 1b and hence, the user can wear the toe stimulus imparting supporter 1b intuitively.

Although preferred embodiments of the toe stimulus imparting supporters 1a, 1b according to the present invention have been described, the present invention is not limited to specific embodiments, and various modifications and changes are conceivable.

What is claimed is:

1. A toe stimulus imparting supporter configured to impart a stimulus to toes in a state where the toes are inserted into the toe stimulus imparting supporter, the toe stimulus imparting supporter comprising:
a body portion formed of an elastic stretchable member having a strip shape;
toe inserting portions through which the toes are insertable, the toe inserting portions formed in both end portions of the body portion in a longitudinal direction of the body portion; and
indirect stimulus imparting protruding portions formed on the both end portions of the body portion in a protruding manner outward from the body portion.

2. The toe stimulus imparting supporter according to claim 1, wherein a direct stimulus imparting protruding portion is formed on an opening edge of each of the toe inserting portions in a protruding manner from at least a front surface or a back surface of the body portion.

3. The toe stimulus imparting supporter according to claim 1, wherein the toe inserting portions are formed such that one toe inserting portion is formed in one end portion of the body portion in the longitudinal direction of the body portion, and two toe inserting portions are formed in the other end portion of the body portion in the longitudinal direction of the body portion.

4. The toe stimulus imparting supporter according to claim 1, wherein an opening which is defined by integrally forming two intermediate portions of the body portion in an intersecting manner is formed as a foot inserting portion.
